(19) United States
(12) Patent Application Publication
(10) Pub. No.: US 2011/0229541 A1
(43) Pub. Date: Sep. 22, 2011

(54) SOLID TEETH BLEACHING DEVICE

(76) Inventors: Philippe Westbroek, Dendermonde (BE); Tamara Van Camp, Overmere (BE); Sander De Vrieze, Eine (BE)

(21) Appl. No.: 13/129,129

(22) PCT Filed: Nov. 13, 2009
(86) PCT No.: PCT/EP09/65166

§ 371 Date: May 13, 2011

(30) Foreign Application Priority Data
Nov. 17, 2008 (GB) 0820938.9

(51) Int. Cl. A61K 8/02; A61Q 11/00

(52) U.S. Cl. 424/401; 424/49; 264/484

(57) ABSTRACT

A teeth bleaching device (100) for bleaching teeth is described being a dry teeth bleaching device, prior to use. The teeth bleaching device (100) comprises a fibrous, non-hydratable polymer structure (110) and a hydratable solid teeth bleaching agent (120) embedded in the fibrous structure prior to use. Upon use, the solid teeth bleaching agent (120) is dissolved by uptake of moisture or saliva in the fibrous structure (110) and released to the teeth. By selecting a predetermined porosity, density and/or fibre diameter, optionally a predetermined dissolving rate and/or release rate of the teeth bleaching agent may be obtained.

### TECHNICAL FIELD OF THE INVENTION

The present invention relates to the field of dental applications. More particularly, the present application relates to methods, products and systems for whitening of teeth.

### BACKGROUND OF THE INVENTION

For bleaching of teeth, two major categories of aesthetic applications are known being applications performed at the dental practice and applications which can be performed outside the dental practice, for example at the consumer's home or at any suitable place. Some of these solutions require a plurality of visits to the dental practice.

Some solutions involving applications which can be performed outside the dental practice, relate to the use of a tray made to fit the mouth and teeth of the user, which is custom-made at the dental practice but which can be used at home. Such a device typically may need to be re-used in view of the cost and must be robust in order to allow repeatedly handling, cleaning, filling, installation, removal, etc.

Low cost solutions also have been provided, wherein a one-size-fits-all system is used. As these systems often do not result in a perfect fit to the teeth, the amount of bleaching agent provided often is increased. On the other hand, such systems also suffer from leakage of the bleaching agent to the gingiva and optionally to ingestion.

US2005/0196352 A1 by the Proctor and Gamble Company discloses a teeth bleaching method whereby the method includes applying a tooth bleaching delivery system to a plurality of adjacent teeth. The tooth bleaching delivery system includes a strip of material and a tooth bleaching composition having a peroxide active component. The method includes applying a first portion of the strip material to the facial surfaces of the teeth and folding a second portion of the strip material over incisal edges of the adjacent teeth to apply the second portion to the lingual surfaces of the teeth. The strip comprises an amount of teeth whitening agent in the form of a gel provided on a polyethylene strip. A disadvantage thereof is that upon handling of the strip for applying it to the teeth, the strip is sticking to the hands and teeth whitening agent is transferred to hands, gum and tongue. The latter typically results in an annoying feeling for the user.

In U.S. Pat. No. 6,780,401 a dry type teeth whitening patch is described. The dry patch comprises a glassy polymer matrix wherein the teeth whitening agent is dispersed. In a dry state, the glassy polymer matrix acts as an impermeable layer for the teeth whitening agent, having little or no adhesive properties. Upon receipt of a significant amount of fluid, the glassy polymer matrix becomes hydrated and allows for the teeth whitening agent to be distributed to the teeth. The hydrated glassy polymer matrix provides an adhesive effect, resulting in sticking of the patch to the surface of the teeth, without the need for further adhesive layer. In order to obtain sufficient stability for the teeth whitening agent, typically a particular stabilizer needs to be added to the teeth whitening agent. Furthermore, good compatibility between the glassy polymer matrix and the teeth whitening agent is to be present.

### SUMMARY OF THE INVENTION

It is an object of the present invention to provide good and save methods and systems for whitening of teeth, also referred to as teeth bleaching. It is an advantage of embodiments according to the present invention that a variety of teeth bleaching agents can be used. It is an advantage of embodiments according to the present invention that methods and systems allow accurate delivery of teeth bleaching agents or components thereof. It is an advantage of embodiments according to the present invention that after dissolving of the solid teeth bleaching agent, the delivery of teeth bleaching agent to the teeth is mainly controlled by structural aspects of the device, more particularly structural aspects of the fibrous structure. It is an advantage of embodiments according to the present invention that the amount of moisture on the teeth and saliva is not the main determining factor in the teeth bleaching agent delivery, after initial dissolving of the solid teeth bleaching agent has taken place. It is an advantage of embodiments according to the present invention that only the teeth bleaching agent and the supporting compounds are dissolvable, not the fibrous structure. The latter results in the fibrous structure being suitable for maintaining its fluid delivery and uptake properties. It thus is an advantage that the structural properties of the fibrous structure may control the delivery and optionally the uptake rather than a change in chemical condition of the polymer forming the structure. It is an advantage of embodiments according to the present invention that methods and systems are provided wherein the uptake of water, e.g. from air, during storage is prevented, therefore requiring little or no stabilisers for stabilising the teeth bleaching agent.

The above objective is accomplished by a method and device according to the present invention.

The present invention relates to a teeth bleaching device, the teeth bleaching device comprising a fibrous, non-hydratable polymer structure and a hydratable solid teeth bleaching agent embedded in, e.g. immobilised by the fibrous structure prior to use. The fibrous structure may be a nano-fibrous structure. By using a nanofibrous structure immobilising of the teeth bleaching agent as well as uptake of moisture and release of teeth bleaching agent once dissolved can be better controlled. The fibrous, non-hydratable polymer structure thereby is a structure comprising fibres of a non-hydratable polymer. It is an advantage of embodiments according to the present invention that methods and systems allow accurate delivery of teeth bleaching agent to the teeth. It is an advantage of embodiments according to the present invention that methods and systems can be provided wherein the teeth whitening and optionally teeth bleaching supporting agents are in solid form to allow a safe and effective and/or efficient teeth bleaching due to limited risk of leaking of the teeth bleaching agent in solid form as well as in liquid form after being dissolved in moist, e.g. saliva, and released by the strip of material. It may for example result in a reduction or even prevention of irritation of parts of the mouth surrounding the teeth.

It is an advantage of embodiments according to the present invention that methods and systems are provided in dry state and as all-in-one systems, thus being user-friendly and allowing more efficient and save methods and systems for whitening of teeth as less handling steps are required.

It is an advantage of embodiments according to the present invention that methods and systems are provided that prevent the teeth bleaching device from leaking the teeth bleaching agent to the area of the mouth surrounding the teeth. The amount of moisture on the teeth and in the mouth thereby does not play a significant role. It is an advantage of embodiments according to the present invention that delivery of teeth whitening agent or components thereof can be provided to all teeth, including the canine teeth and teeth at the backside in the mouth. The latter can be obtained as no folding of the device is required in view of the inherent adhesive properties of the device.

The teeth whitening agent and supporting compounds may for example be immobilized in a solid form in the pores of the fibrous material, immobilised as coatings at the surface of the fibres, immobilized inside the fibers as dissolvable molecules or combinations thereof. The amount of teeth bleaching agent present in solid form can be similar as the amount inserted in the structure as a solution or a gel in a wet teeth bleaching strip. The hydratable solid teeth bleaching agent may comprise teeth bleaching agent crystals embedded in the fibrous structure. The formation of crystals can be easily obtained by providing a fluid version of the teeth bleaching agent to the fibrous structure and by drying it. The fibrous structure may comprise cavities between the fibres adapted to immobilise the teeth bleaching agent crystals. The cavities may be adapted in size to immobilise the teeth bleaching agent crystals. The hydratable solid teeth bleaching agent may comprise a teeth bleaching agent as individual coating on at least part of the individual fibres of the fibrous, non-hydratable polymer structure.

The solid teeth bleaching agent may be a hydrogen peroxide generating product. It may for example be a peracetic acid and carbamide peroxide.

The fibrous structure may be self-supporting, the fibrous structure providing the free surface facing away from the teeth when the teeth bleaching device is positioned on the teeth. It is an advantage of embodiments of the present invention that less components are required resulting in a more efficient and more controllable manufacturing process, while introducing new possibilities for controlling uptake of moisture.

It is an advantage of the embodiments of the present invention that the fibrous structure can be used with or without a backing layer. The latter may result in a more efficient production process of the devices, as less components may be required. It may result in less or no need for additional components such as plasticisers. The latter may also result in more efficient teeth whitening agent delivery to the teeth because the fibrous structure is more open, thus allowing more saliva to enter in a controlled way and direction (preferably towards the teeth).

In some embodiments according to the present invention, the fibrous structure may have a difference in porosity, a difference in density or a difference in fibre diameter over its cross section adapted for providing a controlled uptake of saliva and/or a controlled dissolution rate of teeth bleaching agent and/or a controlled transport and release of the bleaching agent towards the teeth surface. The difference over or along a cross-section may be a difference in depth direction of the fibrous structure. It is an advantage that devices with accurate control of the dissolution rate of the teeth bleaching and/or the release rate of teeth bleaching agent and/or of the resulting bleaching process can be obtained. Control of the dissolution rate of the teeth bleaching agent and therefore control of the teeth bleaching process can be obtained and an efficient teeth bleaching agent delivery can be obtained. In some embodiments according to the present invention, the fibrous structure may have a predetermined profile in porosity, density or fibre diameter over its cross section adapted for obtaining a predetermined uptake profile of saliva and/or release profile of the teeth bleaching agent towards the teeth. It is an advantage of embodiments according to the present invention that a controlled release of the teeth bleaching agent can be provided, assisting in obtaining a homogeneous effect during the treatment and avoiding spike release harming the enamel layer of the teeth.

The porosity of the fibrous structure may increase from the side contacting the backing layer or free side facing away from the teeth during application if there is no backing layer present to the side that will contact the surface of the teeth during application.

The fibrous structure may be a laminated fibrous structure comprising at least two layers of nanofibres having a different diameter.

A teeth bleaching supporting agent such as a pH regulating agent may be provided. It is an advantage of the embodiments of the present invention that the pH regulating agent may also be incorporated in the device in solid form and that it may dissolve together with the teeth bleaching agent. The latter may result in activation of the teeth bleaching system only upon dissolution the pH regulating system and in an efficient regulation of the pH to an optimal value to activate the teeth bleaching agent in a safe and effective manner at the moment of use. The latter is advantageous as it allows to increase the efficiency of the bleaching agent during use and/or it allows to increase the product lifetime of the system.

The pH regulating agent may comprise any or a combination of sodium hydroxide, hydrogen chloride, sodium phosphate buffer system, sodium carbonate buffer system, sodium stannate, citric acid or sodium citrate.

The concentration of bleaching agent may be such that between 2 mg and 530 mg is delivered to the teeth when the device is applied between 2 and 25 minutes, e.g. for application during about 10 minutes. The concentration of bleaching agent may for example be between 0.3 mg/cm² and 80 mg/cm², for a structure with a thickness of 0.1 mm.

The teeth bleaching agent may comprise any or a combination of peroxides or peroxide generating compounds. Peroxides may be for example hydrogen peroxide or calcium peroxide. Peroxide generating compounds may be percarbonates such as for example carbamide peroxide, perborates or peroxyacids. Bleaching materials that advantageously may be used are hydrogen peroxide or carbamide peroxide or a mixture thereof. It is an advantage of carbamide peroxide that it is stable and solid.

In the fibrous structure, at least 30%, preferably at least 50% of the fibres of the fibrous structure may have an average diameter between 3 and 2000 nm.

The fibrous structure, e.g. nano-fibrous structure, may comprise at least 50% of straight fibres wherein the fibres have segments substantially straight over a distance of at least 5 μm.

The fibrous structure, e.g. nano-fibrous structure, may comprise at least 50% of randomly oriented fibres.

The fibrous structure, e.g. nano-fibrous structure, may be an electrospun fibrous structure.

Upon application of the system between 2 and 30 minutes onto the teeth with a frequency of twice a day during a period of between 5 and 14 days, a teeth whitening benefit of at least 1 and maximum 14 shades on the Vitashade® scale may be obtained. It is an advantage of embodiments according to the present invention that these provide in good whitening effects.

The fibrous structure may comprise fibres made of polyamide made by electrospinning using a mixture of formic acid and acetic acid. The ratio of formic acid and acetic acid may be between 90/10 and 10/90 weight percent, preferably between 30/70 and 70/30 weight percent and more preferably between 40/60 and 60/40 weight percent. It may be a 50/50 weight percent ratio.

The teeth bleaching device may be ready for application to at least one tooth and enclosed in a blister. It is an advantage of embodiments according to the present invention that the amount of manipulation required for applying the strip is limited, resulting in a userfriendly device.

The present invention also relates to the use of a teeth bleaching device as described above for teeth bleaching.

The present invention also relates to a nano-fibrous structure, the nano-fibrous structure being adapted for use in a teeth whitening application.

The nano-fibrous structure may be adapted for fitting to one or more teeth, e.g. a row of front teeth. The nano-fibrous structure of the system may be adapted for fitting to incisor teeth and canine teeth, including the tips of the canine teeth. It may also be adapted to fit to other teeth, e.g. positioned at the back of the mouth.

The nano-fibrous structure may be compressible to a thickness between 100 nm and 10 mm. In some embodiments, the nano-fibrous structure may have a thickness between 1 μm and 5000 μm, advantageously between 1 μm and 2000 μm.

The structure of the nano-fibrous structure may be adapted for inducing an adhesive effect of the system on the surface of teeth.

The nano-fibrous structure may have an average porosity between 65 and 99%, preferably between 70 and 98 and more preferably between 75 and 95%. The nano-fibrous structure may have a difference in porosity, difference in density and/or difference in fibre diameter over its cross section adapted for providing a controlled transport of the bleaching agent towards the teeth. The difference over or along a cross-section may be a difference in depth direction of the fibrous structure. The nano-fibrous structure may have a difference in porosity, density and/or fibre diameter over its cross section adapted for providing a controlled uptake of saliva contributing to more efficient release of teeth bleaching agent. The nano-fibrous structure may have a predetermined profile in porosity, density and/or fibre diameter over its cross section adapted for obtaining a predetermined release profile of the bleaching agent towards the teeth. The nano-fibrous structure may have a predetermined profile in porosity, density and/or fibre diameter over its cross section adapted for obtaining a predetermined uptake profile of saliva, optionally resulting in a predetermined transport profile of teeth bleaching agent towards the teeth. The porosity of the nano-fibrous structure may increase from the side contacting the backing layer or the free side to the side that will contact the surface of the teeth.

The nano-fibrous structure may be a laminated nano-fibrous structure comprising at least two layers of nanofibres having a different diameter.

The nano-fibrous structure may comprise fibres made of polyamide made by electrospinning using a mixture of formic acid and acetic acid. The ratio of formic acid and acetic acid may be between 90/10 or 10/90 weight percent, preferably between 30/70 and 70/30 weight percent and more preferably between 40/60 and 60/40 weight percent. It may be 50 weight percent to 50 weight percent.

The fibrous structure may be adapted for fixating teeth whitening and supporting agents in pores of the nanofibrous structure; for having teeth whitening and supporting agents coated on the surface of the nanofibres or for incorporating the teeth whitening and supporting agents inside the nanofibers or a combination thereof.

The present invention also relates to the use of a nano-fibrous structure as described above for teeth bleaching.

The present invention also relates to a method for bleaching teeth, the method comprising applying a teeth bleaching device comprising a fibrous, non-hydratable polymer structure and hydratable solid teeth bleaching agent embedded in, e.g. immobilised by, the fibrous structure prior to use, to at least one tooth, thus initiating a bleaching process of at least one tooth. The teeth bleaching device thereby may be any teeth bleaching device as described above.

It is an advantage of embodiments according to the present invention that user-friendly methods and systems for bleaching of teeth are provided being safer for handling than wet devices, as less or no teeth bleaching agent is transferred to hands or parts of the mouth different from the teeth during application of the strip.

The present invention also relates to a method for manufacturing a nano-fibrous structure, the method comprising electrospinning nano-fibrous structures using a mixture of formic acid and acetic acid. The ratio of formic acid and acetic acid may be between 90/10 or 10/90 weight percent, preferably between 30/70 and 70/30 weight percent and more preferably between 40/60 and 60/40 weight percent. It may be 50 weight percent to 50 weight percent.

The present invention furthermore relates to a method for manufacturing a teeth bleaching device, the method comprising preparing a polymer solution of a non-hydratable polymer in a suitable solvent, electrospinning a fibrous structure of a non-hydratable polymer material, providing a teeth bleaching agent in a liquid phase into the fibrous non-hydratable polymer structure, and converting the liquid phase of teeth bleaching agent in a solid phase by drying the device.

The present invention also relates to a method for manufacturing a teeth bleaching device, the method comprising preparing a polymer solution of a non-hydratable polymer in a suitable solvent, electrospinning a first fibrous structure of a non-hydratable polymer material, providing a teeth bleaching agent in solid phase onto the fibrous structure after said electrospinning a first fibrous structure, and electrospinning a second fibrous structure of a non-hydratable polymer material on top thereof, after said providing a teeth bleaching agent in solid phase so as to sandwich the teeth bleaching agent in solid phase between said first fibrous structure and the second fibrous structure. The teeth bleaching device may comprise more than two fibrous sub-structures between which teeth bleaching agent may be provided by repeating steps as described above.

The present invention furthermore relates to a method for manufacturing a teeth bleaching device, the method comprising preparing a polymer solution of a non-hydratable polymer in a suitable solvent, electrospinning a fibrous structure of a non-hydratable polymer material, and providing, during said electrospinning of the fibrous structure, providing teeth bleaching agent in solid phase so that said solid phase teeth bleaching agent is immobilised through inclusion by the fibres in the fibrous structure.

The present invention furthermore relates to a method for manufacturing a teeth bleaching device, the method comprising preparing a polymer solution of a non-hydratable polymer in a suitable solvent, providing teeth bleaching agent in the solution, and electrospinning a fibrous structure of the mixture of a non-hydratable material and the teeth bleaching agent. The teeth bleaching agent then is solid and captured inside and on top of the fibres that result in the fibrous structure.

Embodiments of the present invention also relate to a combination of any of the methods for manufacturing as described above.

Particular and preferred aspects of the invention are set out in the accompanying independent and dependent claims. Features from the dependent claims may be combined with features of the independent claims and with features of other dependent claims as appropriate and not merely as explicitly set out in the claims.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 shows an example of a diagrammatic representation of a teeth whitening system comprising a fibrous, non-hydratable polymer structure and solid particles of teeth bleaching agent embedded therein, according to an embodiment of the present invention.

FIG. 2 shows an example of a diagrammatic representation of a teeth whitening system comprising a fibrous, non-hydratable polymer structure and a solid coating of teeth bleaching agent on the fibres of the fibrous structure according to an embodiment of the present invention.

FIG. 3 shows an example of a diagrammatic representation of a teeth whitening system comprising a non-hydratable fibrous polymer structure, dissolvable teeth bleaching agent being spun in the fibres of the fibrous non-hydratable polymer structure according to an embodiment of the present invention.

FIG. 4 indicates a SEM picture of a fibrous structure where teeth bleaching agent is present as crystals around the fibres, according to an embodiment of the present invention.

FIG. 5 is an indication of a fibre diameter profile that can be used for controlling the uptake and release of saliva respectively teeth bleaching agent, according to an embodiment of the present invention.

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention. Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein. It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

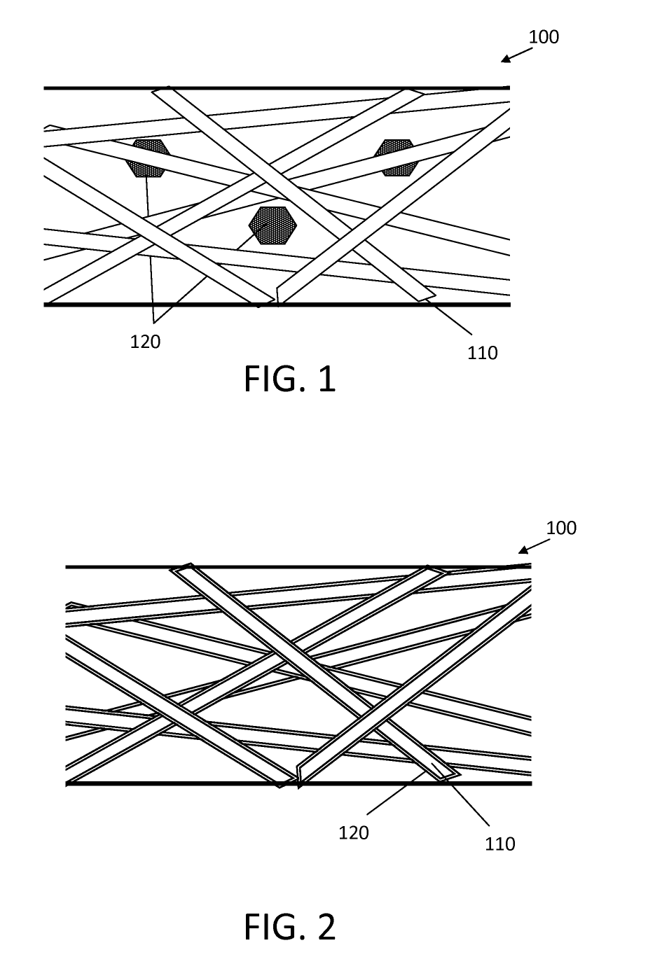

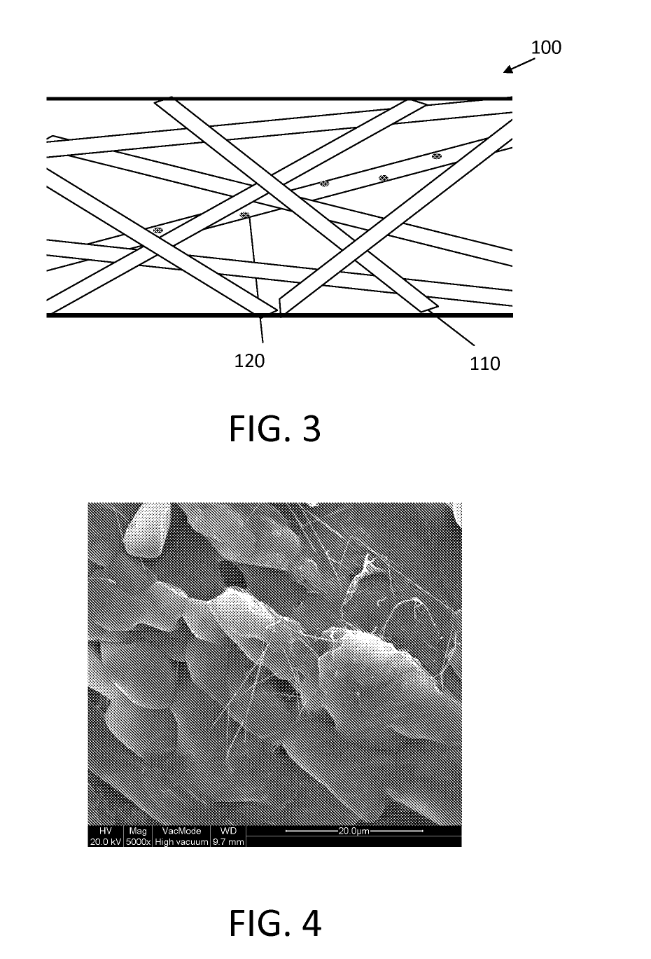

According to a first aspect, the present invention relates to a teeth bleaching device for bleaching teeth, also referred to as whitening teeth. The device also may be referred to as teeth bleaching strip. The teeth bleaching device comprises a non-hydratable fibrous polymer structure. The fibrous, non-hydratable polymer structure may be a non-dissolvable fibrous structure. The fibrous structure advantageously may be a nano-fibrous structure, although the invention is not limited thereto. The teeth bleaching device furthermore comprise hydratable but solid teeth bleaching agent embedded in, e.g. immobilised by, the fibrous structure prior to use. The teeth bleaching agent may be immobilised in its solid form in the fibrous polymer structure. Embedded and optionally immobilised thereby may for example be obtained by the teeth bleaching agent being fixated in the pores of the fibrous structure, e.g. as crystals or crystallites, by the teeth bleaching agent being coated onto the fibres of the fibrous structure, by the teeth bleaching agent being incorporated into the fibres of the fibrous structure, by the teeth bleaching agent being provided as separate fibres of the fibrous structure e.g. by co-spinning it during an electrospinning process of the fibrous structure, or by a combination thereof. In the first two types of embodiments, the fibrous, non-hydratable polymer structure comprises all non-hydratable fibres whereas in the last type of embodiments, additional hydratable fibres, being the teeth bleaching agent fibres, are embedded in the fibrous, non-hydratable polymer structure or hydratable portions are inserted in the fibres of the fibrous, non-hydratable polymer structure. In all embodiments the fibrous non-hydratable polymer structures comprises the majority of fibres, allowing to maintain a basic structure, even after the teeth bleaching agent and optionally further components have been dissolved. The fibrous polymer structure according to embodiments of the present invention does not substantially dissolve, thus allowing to maintain its release and uptake properties. Besides teeth bleaching agent, also teeth bleaching supporting agents may be provided in solid form embedded in the fibrous structure. The fibrous structure thus may act as a carrier for the teeth bleaching and teeth bleaching supporting agents during storage and as a patch releasing these compounds during use. It is an advantage of a teeth bleaching device as described above that before use and during positioning, the system acts as a dry teeth bleaching system, not or substantially not transferring teeth bleaching agent or other agents to the hands or parts of the mouth of the user different from the teeth before actual initiation of the teeth bleaching process. It furthermore is an advantage that the release and uptake of fluids is mainly determined by the structure of the fibrous, non-hydratable polymer structure. By way of illustration FIG. 1 illustrates an example of a teeth bleaching device comprising a fibrous, non-hydratable polymer structure 110 comprising solid teeth bleaching agent 120 embedded in the structure, prior to use. FIG. 2 illustrates another example of a teeth bleaching device comprising a fibrous, non-hydratable polymer structure 110 comprising the solid teeth bleaching agent 120 as coating on the fibres of the fibrous structure. In a third example in FIG. 3, a fiborous, non-hydratable polymer structure 110 is shown, comprising the solid teeth bleaching agent 120 as dissolvable molecules in the fibres of the fibrous polymer structure 110. FIG. 4 illustrates a scanning electron microscope image of a fibrous, non-hydratable polymer structure 110 embedding crystals of teeth bleaching agent formed around the fibres of the structure. The latter is obtained by dispersing the fibrous structure in liquid teeth bleaching agent and drying the structure. The crystals formed in the present example are about 5 micrometer large.

By way of illustration, the present invention not being limited thereto, further features of the components above as well other aspects of the teeth bleaching device as described above will be provided, the present invention not being limited thereby.

The fibrous, non-hydratable polymer structure 110 may optionally have one or more of the properties as described below.

The fibrous structure 110 may comprise fibres such that at least 30%, advantageously at least 50% of the fibres of the fibrous structure have an average diameter between 3 and 2000 nm. The diameter of a majority of the fibres (i.e. 50% or more of the fibers) comprised in the fibrous structures of the present invention may have a diameter of 3 nm or higher, advantageously 10 nm or higher. As an optional feature, the diameter of a majority of the fibres (i.e. 50% or more) comprised in the fibrous structures of the present invention have a diameter of 2000 nm or lower, advantageously 800 nm or lower, most advantageously 700 nm or lower. For the present application, fibres with diameters with one of the suggested diameter or within one of the diameter ranges will be referred to as nanofibres, and the corresponding structures may be referred to as nano-fibrous structures. In embodiments, the present invention also relates to a fibrous structure wherein the thickness of the fibres is uniform, i.e. the standard deviation of the thickness throughout the fibrous structure does not exceed 80%, advantageously 50%, more advantageously 20%. The fibre diameter is dependent on the distance between the outlets and the receiving surface. The profile of the relationship between the fibre diameter and the distance may be polymer and solvent specific. Therefore a profile can be determined after studying the polymer solution or melt because it is polymer and solvent specific. It can be determined via trial and error, via experimental results, via a theoretical model, etc. The individual nanofibres may have a length between 10 μm and 50 m.

The fibrous, non-hydratable polymer structure 110 may comprise non-hydratable fibre material that can be any suitable non-hydratable material, such as for example polyamides, polystyrenes, polyacrylonitriles, polyimides, non-hydratable polyurethanes, cellulose, non-hydratable methacrylates, silk, polyvinylbutyral and metal containing nanofibers. The fibrous structure 110 may comprise fibres made of polyamide made by electrospinning using a mixture of formic acid and acetic acid. The ratio of formic acid and acetic acid may be between 90/10 and 10/90 weight percent, preferably between 30/70 and 70/30 weight percent and more preferably between 40/60 and 60/40 weight percent. It may advantageously be a 50/50 weight percent ratio. It is an advantage of a ratio of around 50/50 weight percent that a steady state continuous production and a high flow rate is possible. As indicated above, in some embodiments, embedded in the fibres of the fibrous non-hydratable polymer structure 110 a teeth bleaching agent, a pH regulating material or a teeth bleaching supporting compound and combinations thereof may be present as dissolvable molecules in the fibres of non-hydratable polymer.

The fibrous structure 110 may have a porosity, e.g. an average porosity, of at least 65%. The porosity, e.g. average porosity, may be between 65 and 99%, advantageously between 70 and 98 and more advantageously between 75 and 95%. The pore sizes can vary from 30 nm to 8 μm. The fibrous structure 110 may for example be able to absorb water, a solution, a compound or a gel for an equivalent of 3 to 12 times its own weight.

The fibrous structure 110 may comprise at least 50% of straight fibres wherein the fibres have segments substantially straight over a distance of at least 5 μm. The straightness of the fibres can for instance be inferred from an image analysis. Preferably, the majority of the fibers (i.e. 50% or more) comprised in the fibrous structure are straight, i.e. consists of a majority of segments (i.e. 50% or more) substantially straight over a distance of 5 μm. By substantially straight, it must be understood that the major axis of the fibre, i.e. along the direction of the fibre, changes over an angle less than 45°, e.g. less than 30°, or e.g. less than 15° or e.g. less than 5°, considering a distance of 10 micrometer over which the angle change was measured. This angle is the largest angle which can be measured between tangents at two points of the major axis over the length of the fiber considered. The standard deviation to linearity over the distance in question may be not exceeding 5%.

As an advantageous feature, the fibrous structure 110 in embodiments according to the present invention may comprise only few or no crosslinking, e.g. it may be micro-fibrous or nano-fibrous structure wherein a majority of the fibers (i.e. 50% or more) comprised are substantially cross-link free. Cross-link free thereby may be that there is absence of covalent bonds linking one polymer chain of one fibre to another polymer chain of a neighbouring fibre. The distance between the three or more outlets also may be adapted for obtaining a fibrous structure comprising at least 50% of fibres substantially free of any chemical bound. They are advantageously not cross-linked to neighboring fibers. Cross-linking thereby means that a link occurs between two fibres, not just that two fibers are touching. This is the result of the spacing between the outlets being at least 1 cm. Without being bound by theory, it is believed that this effect is due to an easier and therefore faster evaporation of the solvent during the fibres formation. It is believed that for spacing between the outlets inferior to 1 cm, the solvent takes too much time to evaporate during the fibres formation. This leads to fusing of adjacent fibres and therefore to crosslinks. If the fibrous structure is made from a melt, the problem may be an incomplete elimination of the heating effect occurring when outlets are too close to each other. The fiber formation then is not complete and a sort of intermediate phase between melt and solid then may be present, allowing formation of cross-linked fibres. Relatively weak physical interactions such as Van Der Waals interactions or hydrogen brigdes are not covered by the definition of cross-links. In some embodiments, although for example non cross-linked fibres are obtained by a spinning process, cross-linking may intentionally or unintentionally occur by a post treatment. Examples of post-treatment can be heating of the nanofibrous structures close to the melting point, or by applying UV irradiation to promote chemical cross linking through grafting principles. The fibrous structure may comprise at least 50% of randomly oriented fibres. In one embodiment, the fibrous structure forms a mat.

Advantageously the fibrous structure 110 may be obtained through electrospinning of a material. Fibres with the features as indicated may be obtained by an electrospinning technique with a multi-nozzle system. The nozzles may be separated at an interdistance of at least 1 cm, e.g. at least 4 cm. Furthermore, for controlling the diameter, the distance between the nozzles and the receiving surface may be adapted. Varying the distance during electrospinning may allow obtaining a variable diameter of the fibres in the fibrous structure. Variation of other parameters for electrospinning in view of the variation of the outlet collector distance, such as for example the voltage to be applied, may for example be determined experimentally.

Turning now to the hydratable but solid teeth bleaching agent 120, the hydratable solid teeth bleaching agent 120 according to embodiments of the present invention and optionally also teeth bleaching supporting agents, are provided in a hydratable but solid form. It may have for example have following features and advantages.

The teeth bleaching agent may be the active component for bleaching a tooth or a plurality of teeth or an agent comprising such a component or able to release or generate such a component. The teeth bleaching agent, sometimes also referred to as bleaching compound, may be a peroxide, such as for example hydrogen peroxide or a hydrogen peroxide generating product, calcium peroxide or a calcium peroxide or a combination thereof. The bleaching agent also may be a peroxide generating compound such as carbamide peroxide, perborates, percarbonates, oxyacids, and/or combinations of these chemicals. The quantity of teeth bleaching agent present in the teeth bleaching device may be between 0.3 mg/cm² and 80 mg/cm². The quantity of teeth bleaching agent present may be such that it results in a delivery of teeth bleaching agent to the teeth of between 2 mg and 530 mg in a period of time between 2 and 25 minutes. The teeth bleaching agent may for example be provided as crystals or crystallites, as dissolvable molecules built in the non-hydratable polymer fibres or as coatings on the fibres of the fibrous structure. Hydratable teeth bleaching agent in embodiments according to the present invention advantageously is dissolvable teeth bleaching agent.

The teeth bleaching supporting agents in solid form may comprise a pH regulation compound, which may be a regular and state of the art standard buffer, acid or base. It may for example be any or a combination of sodium hydroxide, hydrogen chloride, sodium phosphate buffer, sodium carbonate buffer, sodium stannate, citric acid or sodium citrate, the invention not being limited thereto as long as the pH regulating compound can be obtained in a solid form. The pH regulating agent advantageously may be inactive in solid form and becomes active upon dissolution in saliva. This dissolution takes place simultaneously with the dissolution of the teeth bleaching agent, thus upon dissolution it also activates the teeth bleaching agent. The amount of pH regulating system is chosen in that way to obtain a pH between 4 and 10 upon dissolution in saliva. The pH regulating system can (as is the case for the teeth bleaching agent) be fixated in the pores of the fibrous structure, as a coating onto the fibers, included inside the fibers or a combination thereof. Including the pH regulating agent inside the fibrous structure 110 may be obtained by adding the appropriate amount of pH regulating agent to the solution for making the fibrous structure, e.g. the electrospinning solution.

The teeth bleaching supporting agents may also comprise a taste product, in order to promote a good taste when teeth bleaching is performed. Examples of such taste products may for example be spearmint/peppermint. The latter also may be provided in a fixed form, e.g. fixated in the pores of the fibrous structure, as a coating onto the fibers, included inside the fibers or as a combination thereof.

It is an advantage of the embodiments of the present invention that the teeth bleaching and optionally the teeth bleaching supporting agents are incorporated in the device prior to use, resulting in a more user-friendly teeth bleaching device, requiring less handling for applying it. It is an advantage of embodiments according to the present invention that the risk of leaking out of the structure, e.g. on the Gingiva or the Palate in the mouth is limited, reduced or even avoided. The teeth bleaching and optionally the supporting agents may also comprise a Gingiva or Palate protecting component, such as for example a chemical component for protecting the Gingiva or Palate protecting component. Further features and options of the teeth bleaching device 100 may be as described below. The teeth bleaching device 100 may be suitable to be fitted to a plurality of teeth for teeth bleaching. The structural properties of the fibrous structure 110 may be suitable for appropriately absorbing moisture, e.g. saliva, allowing the solid hydratable teeth bleaching agent 120 and optionally other supporting agents to be dissolved. The structural properties of the fibrous structure 110 may be adapted for releasing the teeth bleaching agent to the teeth, once these are dissolved. It is an advantage of embodiments according to the present invention that the fibrous structure of the device advantageously are moisture permeable, e.g. water permeable, thus assisting in dissolving the teeth bleaching agent and in transport of the teeth bleaching agent towards the teeth. The latter allows delivery of teeth bleaching agent, even if little moisture is present. It is an advantage of embodiments according to the present invention that good transport of the teeth bleaching agent from the device to the teeth can be obtained.

According to an embodiment of the present invention, the structure of the fibrous structure can be selected so as to tune or optimise the transport of saliva through the strip of material, as will be described below with reference to a particular embodiment. The latter may allow accurately dissolving the teeth bleaching agent and transport it to the surface of the teeth. The latter results in further improvement of the teeth bleaching efficiency. The amount of moisture present in the mouth thereby plays only a limited role as the transfer may be mainly determined by the structure of the teeth bleaching device, more particularly by the structure of the fibrous structure.

The fibrous structure 110 may be supported by a backing layer, although the invention is not limited thereto. The optional backing layer can be made of any material that is certified for use in the mouth. The backing layer may be substantially water impermeable and water insoluble. It is an advantage of embodiments according to the present invention that the backing layer may provide a barrier for leakage of teeth bleaching agent e.g. to the gingival area, lips and/or at the tongue. It may for example be a material that can adopt to the shape of the teeth such as aluminium foil because it is suitable for nano-fibrous deposition in the electrospinning method and it keeps its shape after bending over the teeth. It is an advantage of embodiments according to the present invention that such a backing layer may provide a better fit of the system over the teeth.

Nevertheless, the fibrous structure 110 also may itself be adapted in structure so that it can keep its shape when provided on the teeth. In advantageous embodiments according to the present invention, the teeth bleaching device may be free of backing layer. The teeth bleaching device therefore may be a strip wherein, during application, the fibrous structure forms the outer surface of the device at the free side facing away from the teeth surface. The fibrous structure may be suitable for keeping the teeth whitening agent from leaving the teeth bleaching device at the free side facing away from the teeth surface. This can be obtained by tuning the density, porosity and/or fibre diameter of the fibrous structure at the surface facing way from the teeth during use. It is an advantage of embodiments according to the present invention that absence of a backing layer can be combined with a porosity profile or fibre diameter profile, in which porosity increases or diameter decreases from the free side to the side contacting the teeth. This may allow controlling the transport direction of saliva taken up from the free side of the device facing away from the teeth surface to the side of the device in contact with the teeth. Teeth bleaching agent will be forced in the same direction resulting in an efficient release towards to teeth. In this way leakage of teeth whitening agent may be avoided without the need for a backing layer. The latter again results in a less complicated and consequently more efficient manufacturing method of teeth bleaching device.

The fibrous structure 110 of the system may be adapted for fitting to a row of front teeth. It is an advantage of embodiments according to the present invention that the fibrous structure is adapted so that it can be compressed to a set of teeth. It is an advantage of using a nano-fibrous structure that the system can maintain a good fit to the teeth. The fibrous structure of the system may be adapted for fitting to incisor teeth and canine teeth, including the tips of the canine teeth. The incisor teeth are the four front teeth at the top or the four front teeth at the bottom of the mouth. It is an advantage of embodiments according to the present invention that the canine teeth adjacent to the incisor teeth also can be bleached, e.g. simultaneously, as these canine teeth often have a more yellowish colour and as bleaching also the canine teeth will result in a more homogeneous colour for canine and incisor teeth so that no large contrast between the canine teeth and the incisor teeth is obtained.

The fibrous structure 110 of the system may also be adapted to a set of teeth larger than or different from the incisor and canine teeth. It is an advantage of the embodiments according to the present invention that more than 6 teeth can be bleached, e.g. simultaneously, because more teeth can be visible during e.g. smiling than only the 6 front teeth. A good fit can be made while allowing to also cover the canine teeth and/or the teeth in the backside of the mouth. It is an advantage of embodiments according to the present invention that the teeth can be whitened in a relative smooth and/or uniform way. In some embodiments according to the present invention, folding of the strip of material over the incisal edges of the teeth can be avoided, while still a good fit can be obtained based on the properties of the nanofibrous structure.

It is an advantage of embodiments according to the present invention that the teeth bleaching device can be easily adapted in length to cover more or less teeth. The latter is further supported by some embodiments where folding of the device over the incisal edges is not required. Optionally, part of a large dry teeth bleaching strip may be cut, so as to fit the teeth that the user wants to bleach.

The fibrous structure 110 may be compressible to a thickness between 100 nm and 10 mm. It is an advantage of embodiments according to the present invention that the device, or at least the fibrous structure thereof, can be pressed to the teeth so that by compressing the device, or at least the fibrous structure thereof, the device or at least the fibrous structure thereof adopts to the shape of the teeth.

The fibrous structure 110 may have a thickness between 1 μm and 5000 μm, advantageously between 1 μm and 2000 μm. The device may have a length between 0.5 and 20.0 cm, more preferably between 2 and 15 cm and most preferably between 4 and 10 cm and a width between 0.3 and 5.0 cm, more preferably between 0.5 and 4 cm and most preferably between 0.8 and 2 cm. In an alternative embodiment the edges of the strip are rounded to avoid possible injuries in the mouth. Other suitable shapes for the device also may be provided.

A perfect fit with the teeth shape can also be obtained because the fibrous structure is a very porous, compressible and fluffy structure, thus surrounds perfectly around the shape of each individual tooth.

The fibrous structure 110 may be adapted in structure for having an adhesive effect of the device on the teeth. It is an advantage of embodiments according to the present invention that a system is provided allowing stable positioning and/or fixation of the device on the teeth of a user. It is an advantage of embodiments according to the present invention that methods and systems are provided requiring less or no separate components for fixing the device to the teeth and/or requiring less or no additional requirements on the properties of the components used. According to embodiments of the present invention, folding of the teeth bleaching device over the incisal edges of the teeth may be done but is not required. The fibrous structure may be responsible for an adhesive effect between the teeth bleaching system and the surface of the teeth based on its high contact surface area. It is an advantage of embodiments according to the present invention that the strip of material may have at least some adhesive properties on itself and therefore can be fixated at the surface of the teeth. It is an advantage of embodiments according to the present invention that the porosity of the fibrous structure is adapted for assisting in the adhesive effect, especially if interaction occurs between the fibrous structure and some moisture on the teeth. The porosity of the fibrous structure in combination with some moisture on the teeth may allow good adhesive effect so that the an additional adhesive material becomes obsolete and thus can be avoided. The fibrous structure thereby may for example be a nano-fibrous structure, although the invention is not limited thereto.

By way of illustration, the present invention not being limited thereto, a particular embodiment with particular advantages is described below.

In one particular embodiment according to the present invention, the fibrous structure 110 may have a difference in porosity, diameter of the fibres or density in one or more dimensions, such as for example in the depth direction of the fibrous structure, i.e. over the cross-section, and/or in the surface direction of the nano-fibrous structure, i.e. in a plane along the surface of the fibrous structure which is suitable for contacting with the teeth. The difference of the porosity, diameter of the fibres or density of the fibres may result in a variation according to a predetermined profile in order to provide a predetermined release profile of the teeth bleaching agent and/or thereof predetermined uptake and/or transport of saliva respectively by and through the nanofibrous material.

In one example, the porosity of the nano-fibrous structure may increase from the side contacting the backing or free side layer to the side that will contact the surface of the teeth. Variation in porosity, diameter of the fibres or density of the fibres may be obtained continuously or stepwise. In one example, a difference in porosity, fibre diameter or fibre density may be obtained by using a laminated fibrous structure comprising at least two layers of nanofibres having a different diameter. Such a laminated fibrous structure may comprise a number of layers wherein discrete steps of a material property are present or there may be a continuous or semi-continuous variation of a material property. Other profiles also may be provided. For example, a profile allowing release of a large portion (e.g. about 40% to 50%) of the bleaching agent may be provided during an initial period, e.g. the first 4 minutes, while the remaining portion may be released over a larger period of time, e.g. the following 9 minutes, subsequent to the initial period.

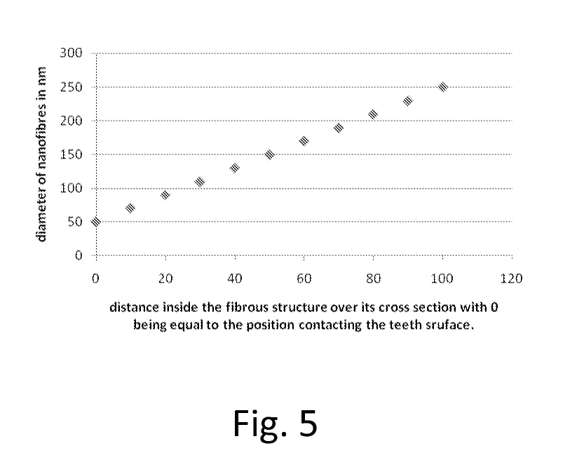

Providing a predetermined profile in density, porosity or fibre diameter thus may provide a predetermined rate of release of the teeth bleaching agent, which may be advantageous for providing a treatment according to a predetermined profile, e.g. with a uniform bleaching agent release. A predetermined profile in density, porosity or fibre diameter may also provide a predetermined rate of uptake of saliva, indirectly determining the dissolution rate of the teeth bleaching and optionally of the teeth bleaching supporting agents and thus also determining their transport and release rate towards the teeth surface. By way of illustration, the present invention not being limited thereto, an exemplary profile for a fibre diameter as function of the depth in the fibrous structure according to an exemplary device is shown in FIG. 5. In the profile shown, the diameter of the fibre increases with the distance to the surface contacting the teeth in use.

In one embodiment, the present invention also relates to a teeth bleaching device as described above, being ready for use by application to at least one tooth, the teeth bleaching device being enclosed in a blister. It is an advantage of embodiments according to embodiments of the present invention that the bleaching device can be packaged ready for use and that additional manipulation can be limited. The blister may be adapted to prevent moisture to enter the blister.

In another aspect, the present invention relates to a method for performing teeth bleaching. The method according to embodiments of the present invention may comprise using, for performing the teeth bleaching, a teeth bleaching device comprising a fibrous, non-hydratable polymer structure and a hydratable solid teeth bleaching agent embedded in, e.g. immobilised by, the fibrous structure. The fibrous structure may be a nano-fibrous structure. Optionally also teeth bleaching supporting agents may be present in solid form in the teeth bleaching device. The method may be applied in any suitable place, e.g. at the consumer's choice, i.e. not being limited to the dental practice but for example also at home. The method has the advantage that it provides bleaching of teeth, which results in an aesthetic effect. The method may be performed using a teeth bleaching system as described in any of the embodiments according to the first aspect of the present invention, although the invention is not limited thereto. As the teeth whitening system according to embodiments of the present invention can be an all-in-one teeth bleaching application, the teeth whitening system may be packed in one single package. The method according to embodiments of the present invention, comprises applying a teeth bleaching device comprising a fibrous, non-hydratable polymer structure and hydratable solid teeth bleaching agent embedded in the fibrous structure prior to use, to at least one tooth, thus initiating a bleaching process of the at least one tooth. The bleaching process thus may be initiated automatically by dissolving of the hydratable solid teeth bleaching agent when moisture at the teeth or in the mouth enters the fibrous structure. The at least one tooth may be a plurality of teeth, e.g. adjacent teeth. The method furthermore may comprise removing the device after a certain application time between 2 and 25 minutes. This procedure is repeated between 7 to 28 times spread over a period of 4 to 14 days.

By using a fibrous structure, the shape of the structure can be appropriately adapted to the shape of the teeth. The fibrous structure may have a structure so that fixation to the teeth is obtained through adhesive properties of the structure, e.g. due to the surface area of the fibres of the fibrous structure that can be in contact with the teeth. Applying the fibrous structure may comprise pressing the nano-fibrous structure at the front side of the teeth. The use of a fibrous structure also has the advantage that a homogeneous bleaching is obtained, whereby a plurality of teeth can be bleached, such as for example both the incisor teeth and the canine teeth at the same time, the invention not being limited thereto. In some embodiments according to the present invention, the fibrous structure may be adapted for dissolving and/or releasing teeth whitening agent in a controlled way, e.g. at a controlled flow rate, to the teeth. The latter may for example be obtained by using a fibrous structure with a varying diameter, porosity or density profile of the fibres used. With this profile release of teeth whitening agent can be controlled directly but also indirectly through controlled uptake and transport rate of saliva. The methods according to embodiments of the present invention furthermore may comprise one or more steps expressing the functionality of one or more components of the teeth whitening system as described in the first aspect. As in its initial state, before use, the device is dry, handling the device, such as for example pressing it against the teeth, does not result in teeth bleaching agent being transferred (substantially) towards the hands of the user.

By way of illustration, the present invention not being limited thereto, an exemplary method according to an embodiment of the present invention is described below. A method for whitening teeth thereby is illustrated, the method making use of a teeth whitening system as described in the first aspect. The teeth whitening system thereby comprises a fibrous, non-hydratable polymer structure, in the present example being deposited on a backing layer, although the invention is not limited thereby. The teeth bleaching device also comprises teeth bleaching agent and optionally teeth bleaching supporting agents. The amount of teeth bleaching agent thereby is between 0.1 and 1 g. In the present example, the teeth bleaching system is sealed in a single package.

According to the exemplary method, the sealed package may be opened and the fibrous, non-hydratable polymer structure comprising the hydratable teeth bleaching agent and optionally supporting agents in solid form may be removed from the sealed package. The fibrous structure then may be positioned onto the teeth by a slight pressure. This positioning may include covering the incisor teeth as well as the canine teeth, including their tips. An adhesive effect of the bleaching device to the teeth may be obtained by the structure of the fibrous structure, without the need for adding an adhesive. The adhesive effect thereby may be enforced by the combination of moisture and the fibrous structure. The fibrous structure deposited on a backing layer is maintained at the teeth for a predetermined time. After treatment, the fibrous structure deposited on a substrate may be removed by hand and the teeth may be rinsed with water or brushed.

In further aspects, the present invention also relates to the use of a teeth bleaching device according to any of the embodiments of the aspects described above in a teeth whitening application. The advantages and features of the systems and/or structures described thereby may result in advantageous use of such systems and/or structures.

In another aspect, the present invention also relates to a method for manufacturing dry teeth bleaching devices. A plurality of methods for manufacturing teeth bleaching devices, e.g. devices as described in the first aspect, are provided. By way of example, the present invention not being limited thereto, a number of particular examples are described in more detail.

In a first particular example, a method for manufacturing a teeth bleaching device is described, wherein the method comprises preparing a polymer solution of a non-hydratable polymer in a suitable solvent, electrospinning a fibrous structure of a non-hydratable polymer material, providing a teeth bleaching agent in a liquid phase into the fibrous non-hydratable polymer structure, and converting the liquid phase of teeth bleaching agent in a solid phase by drying the device. Electrospinning thereby may be performed using a single or advantageously multi-nozzle system. It may for example be performed at an applied voltage between tip of the nozzles and the collector surface between 1000 and 3000 V cm⁻¹. The polymer solution used may for example contain 12% polyamide in 50/50 weight % formic acid and acetic acid. A polymer solution feeding rate between 0.3 and 15 mL h⁻¹ and an ambient humidity between 10 and 90% may be used. The teeth bleaching agent provided in liquid phase may result in a teeth bleaching agent present in a concentration between 0.3 and 80% in the fibrous structure. It may be provided in the fibrous structure through impregnation with the liquid phase or by dipping the fibrous structure in the liquid phase. Drying the device may be performed at a temperature between 30 and 80 degrees Celcius. The drying process can be done open in the air, with an air stream or in an oven. The resulting solid form of the teeth bleaching agent may be in the form of crystals present in the pores of the fibrous structure.

In a second particular example, a method for manufacturing a teeth bleaching device is described comprising preparing a polymer solution of a non-hydratable polymer in a suitable solvent, electrospinning a first fibrous structure of a non-hydratable polymer material, providing a teeth bleaching agent in solid phase onto the fibrous structure after said electrospinning a first fibrous structure, and electrospinning a second fibrous structure of a non-hydratable polymer material on top thereof, after said providing a teeth bleaching agent in solid phase so as to sandwich the teeth bleaching agent in solid phase between said first fibrous structure and the second fibrous structure. The teeth bleaching device may comprise more than two fibrous sub-structures between which teeth bleaching agent may be provided by repeating steps as described above. Electrospinning thereby may be performed using a single or advantageously multi-nozzle system. It may for example be performed at an applied voltage between tip of the nozzles and the collector surface between 1000 and 3000 V cm⁻¹. The polymer solution used may for example contain 12% polyamide in 50/50 weight % formic acid and acetic acid. A polymer solution feeding rate between 0.3 and 15 mL h⁻¹ and an ambient humidity between 10 and 90% may be used. The method may result in a non-continuous layer of teeth bleaching particles being sandwiched between two fibrous structures of non-hydratable polymers.

In a third particular embodiment, a method for manufacturing a teeth bleaching device is described, the method comprising preparing a polymer solution of a non-hydratable polymer in a suitable solvent, electrospinning a fibrous structure of a non-hydratable polymer material, and providing, during said electrospinning of the fibrous structure, providing teeth bleaching agent in solid phase so that said solid phase teeth bleaching agent is immobilised through inclusion by the fibres in the fibrous structure. Electrospinning thereby may be performed using a single or advantageously multi-nozzle system. It may for example be performed at an applied voltage between tip of the nozzles and the collector surface between 1000 and 3000 V cm⁻¹. The polymer solution used may for example contain 12% polyamide in 50/50 weight % formic acid and acetic acid. A polymer solution feeding rate between 0.3 and 15 mL h⁻¹ and an ambient humidity between 10 and 90% may be used. The solid teeth bleaching agent can for example be mixed with the nanofibres through an air stream in which the teeth bleaching agent is brought and mixed with the polymer solution jet streams that are simultaneously transformed into nanofibres. After the introduction of solid teeth bleaching agent during the electrospinning, an additional non-hydratable polymer layer may be spun on top thereof.

In a fourth particular embodiment, a method for manufacturing a teeth bleaching device is described, the method comprising preparing a polymer solution of a non-hydratable polymer in a suitable solvent, providing teeth bleaching agent in the solution, and electrospinning a fibrous structure of the mixture of a non-hydratable material and the teeth bleaching agent. The teeth bleaching agent then is solid and captured inside and on top of the fibres that result in the fibrous structure. The electrospinning may be peformed at an applied voltage between tip of the nozzles and the collector surface between 1000 and 3000 V cm⁻¹. The polymer solution used may for example contain 12% polyamide in 50/50 weight % formic acid and acetic acid. A polymer solution feeding rate between 0.3 and 15 mL h⁻¹ and an ambient humidity between 10 and 90% may be used.

Embodiments of the present invention also relate to a combination of any of the methods for manufacturing as described above.

Whereas particular examples have been described above, it will be clear that the processing conditions are only provided by way of illustration, embodiments of the present invention not being limited thereto.

By way of illustration, embodiments of the present invention not being limited thereto, an example according to a particular embodiment is shown below.

In the example given, the teeth bleaching device comprises a fibrous structure of polyamide 6, having dimensions of 8 cm by 1 cm, in which a teeth bleaching agent being carbamide peroxide is immobilized in solid form. The device is positioned onto the front surface of a number of adjacent teeth by a slight pressure. The device is maintained at the teeth for a period of about 10 minutes. After treatment the device is removed by hand and the teeth are rinsed with water and/or brushed. This process is repeated twice a day for example for seven days.

The device as described above was used by 5 test persons according to the procedure described above. All test persons started with a teeth colour corresponding to A₃ on the VITA® shade scale. After 7 days of treatment the VITA shade of all teeth improved to A₁ shade, which is visually much more white than A₃. After 6 months the A₁ value is still maintained.

A comparative test also was performed between the above described strip, further referred to as dry strip, with a commercial gel-based teeth bleaching strip. The commercial strip is a polyethylene film on which an amount of gel was deposited, the gel containing hydrogen peroxide. The strip was applied to the teeth with the side covered with gel applied to the front side of the teeth. The amount of peroxide present in the gel of the commercial strip is identical to the amount of peroxide present in the dry strip. For each product a test group of 10 persons was used. Each person applied 2 strips per day. The whitening effect on the colour of the teeth was determined using the VITA® shade scale. The application time for the dry strip was 10 minutes per application, while for the gel-based teeth bleaching strip the application time per application was 30 minutes, as prescribed by the producer. Both strip types were applied for 14 days.
Whereas the maximum bleaching effect was reached by 90% of the dry strip users, this was only 60% for the gel-based teeth bleaching strip users. Whereas the final bleaching effect was already reached after 7 to 9 days when using the dry strip, 12 to 14 days were required when using the commercial strip. Furthermore, whereas 30% of the gel-based teeth bleaching strip users experienced irritation of the gum, no gum irritation could be identified in the dry strip user group. The dry strip was, by the dry strip users, indicated as being user friendly, especially as it did not move over the teeth during use and as there was no need for a sticky gel. The application of the dry strip was experienced as simple, whereas this was not the case with the commercial strip because the commercial strip was thin and the gel stick to the hands.
The above comparison shows that the dry strip provides good efficiency allowing short application times and short application periods (number of applications required). The short application time is experienced as strong advantage by the user group.
Furthermore, the above tests show that the dry strip provides safety of use, as gum irritation is avoided.

1-17. (canceled)

18. A teeth bleaching device comprising:
a fibrous, non-hydratable polymer structure, and
a hydratable solid teeth bleaching agent embedded in the fibrous structure prior to use.

19. The teeth bleaching device according to claim 18, wherein the hydratable solid teeth bleaching agent is immobilised by the fibrous structure prior to use.

20. The teeth bleaching device according to claim 18, wherein the fibrous, non-hydratable polymer structure is a nano-fibrous structure.

21. A teeth bleaching device according to claim 18, wherein the hydratable solid teeth bleaching agent comprises teeth bleaching agent crystals embedded in the fibrous structure.

22. The teeth bleaching device according to claim 21, wherein the fibrous structure comprises cavities between the fibres adapted to immobilise the teeth bleaching agent crystals.

23. The teeth bleaching device according to claim 22, wherein the cavities are adapted in size to immobilise the teeth bleaching agent crystals.

24. The teeth bleaching device according to claim 18, wherein the hydratable solid teeth bleaching agent comprises a teeth bleaching agent as separate or individual coating on at least part of the individual fibres of the fibrous, non-hydratable polymer structure.

25. The teeth bleaching device according to claim 18, wherein the hydratable solid teeth bleaching agent is embedded in the fibres of nonhydratable polymer as dissolvable molecules.

26. The teeth bleaching device according to claim 18, wherein the fibrous structure is self-supporting, the fibrous structure providing the free surface facing away from the teeth when the teeth bleaching device is positioned on the teeth.

27. The teeth bleaching device according to claim 18, wherein the fibrous, non-hydratable polymer structure comprises a difference in fibre density, a difference in porosity or a difference in diameter of fibres, along a cross-section of the fibrous structure so as to control an uptake of saliva thus controlling a dissolution rate of the teeth bleaching agent.

28. The teeth bleaching device according to claim 18, wherein the fibrous, non-hydratable polymer structure comprises a difference in fibre density, a difference in porosity or a difference in diameter of fibres along a cross-section of the fibrous structure so as to control a release of teeth bleaching agent to the teeth when the teeth bleaching device is positioned on the teeth.

29. The teeth bleaching device according to claim 18, wherein the teeth bleaching device comprises furthermore hydratable but solid teeth bleaching supporting agents, prior to use.

30. The teeth bleaching device according to claim 29, wherein the hydratable but solid teeth bleaching supporting agent comprises a pH regulating agent.

31. The teeth bleaching device according to claim 18, the teeth bleaching device being ready for application to at least one tooth, enclosed in a blister.

32. A method for bleaching teeth, the method comprising the steps:
applying a dry teeth bleaching device comprising a fibrous, non-hydratable polymer structure with hydratable solid teeth bleaching agent immobilised by the fibrous structure prior to use, to the surface of at least one tooth to be bleached, thus initiating a teeth bleaching process.

33. The method according to claim 32, wherein applying a dry teeth bleaching device comprises applying the teeth bleaching device recited in claim 18.

34. A method for manufacturing a teeth bleaching device, the method comprising the steps:
preparing a polymer solution of a non-hydratable polymer in a suitable solvent,
electrospinning a fibrous structure of a non-hydratable polymer material,
providing a teeth bleaching agent in a liquid phase into the fibrous non-hydratable polymer structure, and
converting the liquid phase of teeth bleaching agent into a solid phase by drying the device.
